(19) United States
(12) Patent Application Publication
(10) Pub. No.: US 2015/0065772 A1
(43) Pub. Date: Mar. 5, 2015

(54) Chemical Process for Hydrogen Separation

(71) Applicant: Ceramatec, Inc., Salt Lake City, UT (US)

(72) Inventor: Ashok V. Joshi, Salt Lake City, UT (US)

(21) Appl. No.: 14/470,642

(22) Filed: Aug. 27, 2014

Related U.S. Application Data
(60) Provisional application No. 61/872,040, filed on Aug. 30, 2013.

(51) Int. Cl. C07C 2/76

(52) U.S. Cl. CPC C07C 2/76
USPC 585/415

(57) ABSTRACT

A process is disclosed for removing hydrogen gas that is produced during a DHA (dehydroaromatization) reaction that is used to produce benzene from methane. The hydrogen gas is reacted with a quantity of an alkali metal to produce an alkali metal hydride, which may be separated out from the benzene and any unreacted methane. This removal of the hydrogen gas “drives” the reaction to produce more benzene, thereby increasing the theoretical yield of the DHA reaction.

### CROSS-REFERENCE TO RELATED APPLICATIONS

This application claims the benefit of U.S. Provisional Application No. 61/872,040, filed Aug. 30, 2013, entitled “Chemical Process For Hydrogen Separation” the entire disclosure of which is hereby incorporated by reference.

### TECHNICAL FIELD

The present application relates to producing benzene (C₆H₆) from methane (CH₄). More specifically, the present process involves processes of removing hydrogen gas from the methane-to-benzene conversion reaction, thereby driving the reaction to produce more benzene (and thereby increasing the yield of benzene).

### BACKGROUND

Currently, petroleum crude costs 6-8 times more than natural gas on an energy content basis. (Natural gas contains large quantities of methane.) Moreover, approximately 97% of natural gas is currently produced from U.S. domestic sources, whereas more than 50% of the crude oil supply is imported into the U.S. This disparity in the production of crude oil has lead to (1) a reduction in petroleum crude usage as well as (2) the emergence of new processes with more attractive economics for producing value-added chemicals and fuels (such as crude oil) from natural gas.

Benzene, which is currently produced from crude oil, is a chemical of great industrial importance with current global consumption at 30 million metric tons per annum and net growth of 4% annually, leading to a total market size of greater than $50 Billion. Benzene is a starting material for Nylons, polycarbonates, polystyrene, epoxy resins and other desirable chemicals. Also, benzene can be directly converted to aniline, chlorobenzene, maleic anhydride and succinic acid. Benzene is also a gasoline component and can be converted to cyclohexane, another gasoline component via a commercial process.

Benzene can be synthesized from natural gas in a single step via a dehydroaromatization (DHA) route. This dehydroaromatization process that produces benzene from natural gas (in a single step conversion process in the absence of oxygen) is summarized as follows.

6CH₄→C₆H₆+9H₂

While the DHA process is commercially very attractive, there are two primary technical commercialization challenges for this reaction:
(1) Kinetic—a coking reaction also occurs on the catalyst surface which competes with the DHA reaction; and
(2) Thermodynamic: Equilibrium conversions of the DHA reaction are limited to ˜12% at 700° C. and 1 atmosphere.

Solving the kinetic challenge associated with the DHA process requires a highly active and benzene selective (e.g., coke resistant) catalyst. Overcoming the thermodynamic limitation requires continuous selective separation/removal of hydrogen at the reaction temperatures. If hydrogen is continuously removed from the reaction products, up to 100% single-pass conversion of the methane into benzene becomes ultimately possible in such a reaction from the thermodynamic vantage point. Commercialization potential of this innovative process will be thereby dramatically improved by overcoming these challenges and more particularly, by finding a hydrogen separation process that will increase the thermodynamic yield of benzene from the DHA reaction. Such a hydrogen-removal process is disclosed herein.

### SUMMARY

This invention involves methods for separating hydrogen out of the benzene/hydrogen products that are formed from the DHA reaction. In some embodiments, this separation may involve adding an alkali metal, such as sodium, lithium, potassium or alloys of these metals (including an Na—Al or Li—Al alloy) to the products. This added alkali metal may react with the produced hydrogen gas, thereby forming an alkali metal hydride. The benzene product as well as the remaining starting material (methane) does not react with the alkali metal. Once reacted with the alkali metal, the alkali metal hydride may easily be separated out from the benzene and the methane. At this point, the remaining methane starting material may be returned to the reaction vessel (for further reacting) and the benzene may be collected. By removing the supply of hydrogen, the reaction is “driven” to produce more products (e.g., more hydrogen and more benzene), and thus the theoretical yield of the DHA reaction increases. Instead of producing only 12% benzene, theoretical yields of up to 30% may be obtained.

### BRIEF DESCRIPTION OF THE SEVERAL DRAWINGS

In order that the manner in which the above-recited and other features and advantages of the invention are obtained and will be readily understood, a more particular description of the invention briefly described above will be rendered by reference to specific embodiments thereof that are illustrated in the appended drawings. Understanding that the drawings depict only typical embodiments of the invention, are not necessarily drawn to scale, and are not therefore to be considered to be limiting of its scope, the invention will be described and explained with additional specificity and detail through the use of the accompanying drawings in which:

FIG. 1 depicts a schematic diagram of a representative embodiment of a device that may be used to separate hydrogen from a chemical reaction;

FIG. 2 depicts schematic diagram of another representative embodiment of a device that may be used to separate hydrogen from a chemical reaction; and

FIG. 3 depicts schematic diagram of another representative embodiment of a device that may be used to separate hydrogen from a chemical reaction.

### DETAILED DESCRIPTION OF THE INVENTION

The present embodiments relate to chemical processes that may be used to remove hydrogen that is produced in the DHA reaction, which in turn, will “drive” the DHA reaction to produce more products (e.g., more hydrogen and more benzene). Thus, by “driving” the reaction to produce more products, more benzene from the DHA reaction will be produced. Thus, the overall theoretical yield for the DHA reaction may be increased from 12% to about 30%.

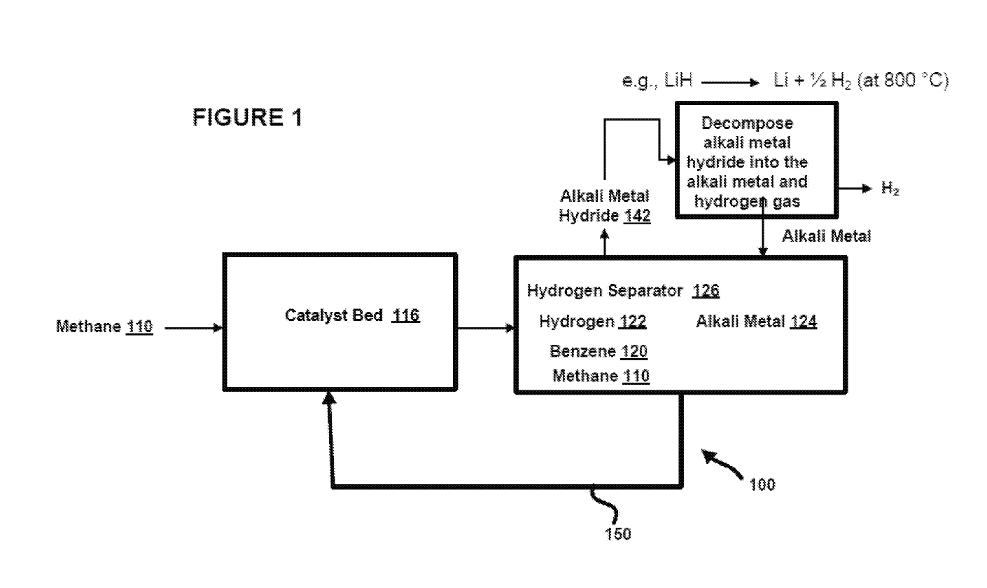

Referring now to FIG. 1, a process 100 is illustrated which uses the DHA reaction to produce benzene 120 and hydrogen gas 122. Specifically, a supply of methane gas 110 is reacted, in the presence of a catalyst bed 116, according to the DHA reaction. As noted above, this reaction produces the benzene 120 and the hydrogen 122 (although the theoretical yield of this reaction is low.) Those skilled in the art will appreciate the various catalysts that may be used in the catalyst bed 116 to facilitate the DHA reaction. One example of a catalyst that may be used is disclosed in U.S. patent application Ser. No. 14/090,776 filed on Nov. 26, 2013, which application is expressly incorporated herein by reference. Other known catalysts for the DHA reaction may also be used.

The products of the DHA reaction include benzene 120 and hydrogen 122. Mixed with these products may be a quantity of unreacted methane 110 (e.g., the starting material). These products, as shown by FIG. 1, may be added to a hydrogen separator 126. In this hydrogen separator 126, a quantity of an alkali metal 124 is added. In some embodiments, the alkali metal 124 may be sodium metal, lithium metal, or potassium metal. Alloys of sodium and lithium, including alloys such as Na—Al and Li—Al may also be used. The alkali metal 124 reacts with the hydrogen 122 to produce an alkali metal hydride 142. The alkali metal 124 does not, however, react with the benzene 120 or the methane 110. Once produced, the alkali metal hydride 142 may be separated out from the products, thereby removing a quantity of the produced hydrogen 122. Of course, this removal of the hydrogen operates to “drive” the reaction to produce more hydrogen (and more benzene), thereby upping the yield of the reaction. Further, as shown by FIG. 1, the benzene 120 may be removed from the hydrogen separator 126 (if desired) and the unreacted methane 110 may be returned for further reaction at the catalyst bed 116, as shown by arrow 150. (Alternatively, both the benzene 120 and the methane 110 may both be returned to the catalyst bed 116.) In some embodiments, only the hydrogen is allowed into the separator 126.

It should be noted that, in some embodiments, the methane 110 used in the DHA reaction may be mixed with other organic gases and/or hydrogen gas. In some embodiments, the hydrogen used in the catalyst bed/separator may be dried to remove moisture. Alternatively, the methane may be completely replaced by another organic starting material that reacts to form benzene 120 and hydrogen 122.

In some embodiments, the hydrogen separator 126 may be a container of molten alkali metal. For example, if the alkali metal is sodium, then the separator 126 may be a container of molten sodium metal that is maintained at a temperature of 200-400° C. In this embodiment, the mixture of products (e.g., the benzene 120, hydrogen 122 and/or the remaining starting material 110) may be bubbled through the container of molten alkali metal, thereby causing the hydrogen to react with the alkali metal to form the alkali metal hydride 142. In other embodiments, only the hydrogen gas 122 (and not the benzene 120 and/or the methane 110) is bubbled through the molten alkali metal.

Once the alkali metal hydride 142 is formed, it may be collected and separated from the other reaction products. Further, the alkali metal hydride may then be heated to dissociate the material to recapture the alkali metal (for further re-use) and obtain hydrogen gas. For example, if the alkali metal is sodium, the sodium hydride is heated to about 800° C., thereby dissociating the material into sodium metal and hydrogen gas. Likewise, if the alkali metal is lithium, the lithium hydride may be heated to temperatures between 800-1200° C., thereby dissociating the material into lithium metal and hydrogen gas. If an alloy of an alkali metal is used, then similar heating temperatures may be required to dissociate the material into the metal and the hydrogen gas. Once the alkali metal and the hydrogen gas have been split (dissociated) via heating, these materials may be cooled to at or below or near the vapor temperature of the alkali metal. If such cooling occurs, the alkali metal returns to its liquid (molten) state, whereas the hydrogen gas remains as a vapor (gas). In this manner, the hydrogen gas may be separated from the alkali metal and the molten alkali metal may be re-used. For example, sodium metal has a boiling point of about 883° C. If the alkali metal hydride was heated to about 850° C., it may dissociate and the sodium will liquefy and can be separated from the gaseous hydrogen gas. Likewise, the boiling point of lithium is about 1342° C. Thus, if the material (or even the hydrogen separator) is maintained at a temperature between about 181-1342° C., then the alkali metal will likely be in its liquid state and can be easily separated from the hydrogen gas (after it dissociates from the hydride form).

It should be noted that the use of the hydrogen separator 126 is not limited to the DHA reaction. Rather, any reaction which produces hydrogen 122 as a product, or for which hydrogen gas may interfere with the catalyst/reaction, may use a hydrogen separator 126 as a means of “trapping” the hydrogen 122 and pushing the reaction to produce the desired products. Moreover, U.S. Provisional Patent Application Ser. No. 61/731,397 filed on Nov. 29, 2012 has additional ways (e.g., via membranes) to extract/trap the hydrogen that is produced in the DHA reaction. Those skilled in the art will appreciate than any of the embodiments shown in this provisional related to removing hydrogen gas (including the use of various membranes) may be incorporated into the present embodiments.

In some embodiments, the hydrogen gas 122 in the mixture of benzene 120/methane 110 mixture is needed in order to stabilize the benzene 120. Accordingly, in these embodiments, not all of the hydrogen gas 122 is removed. However, other embodiments may be designed in which all of the hydrogen gas 122 is removed from the products.

As noted above, embodiments may be constructed in which the hydrogen separator 126 comprises a vessel that houses molten alkali metal 124. In other embodiments, the hydrogen separator 126 is a vessel in which the alkali metal is added to a ceramic powder, and the hydrogen gas is passed through the ceramic powder, thereby causing the alkali metal to react with the hydrogen gas. Other methods of contacting the alkali metal with the hydrogen gas may also be used within the hydrogen separator 126.

Further, embodiments may be designed in which the alkali metal comprises an alloy of sodium or lithium, including a Li—Al alloy or a Li—Si alloy. The lithium-aluminum alloy may be particularly suitable, in some embodiments, because this alloy has a melting point over 500° C. Thus, this particular alloy will be solid over a wide range of temperatures. Of course, other alloys of the alkali metal may also be used.

As shown in FIG. 1, once the alkali metal hydride 142 is formed, this material may be further reacted. For example, it may be placed in a reactor and then decomposed into its constituents. This reaction may occur at elevated temperatures, such as, for example, 800° C. or higher. In the embodiment shown in FIG. 1, lithium hydride is decomposed into lithium metal and hydrogen gas. The hydrogen gas may be collected and the lithium metal then added back into the separator 126.

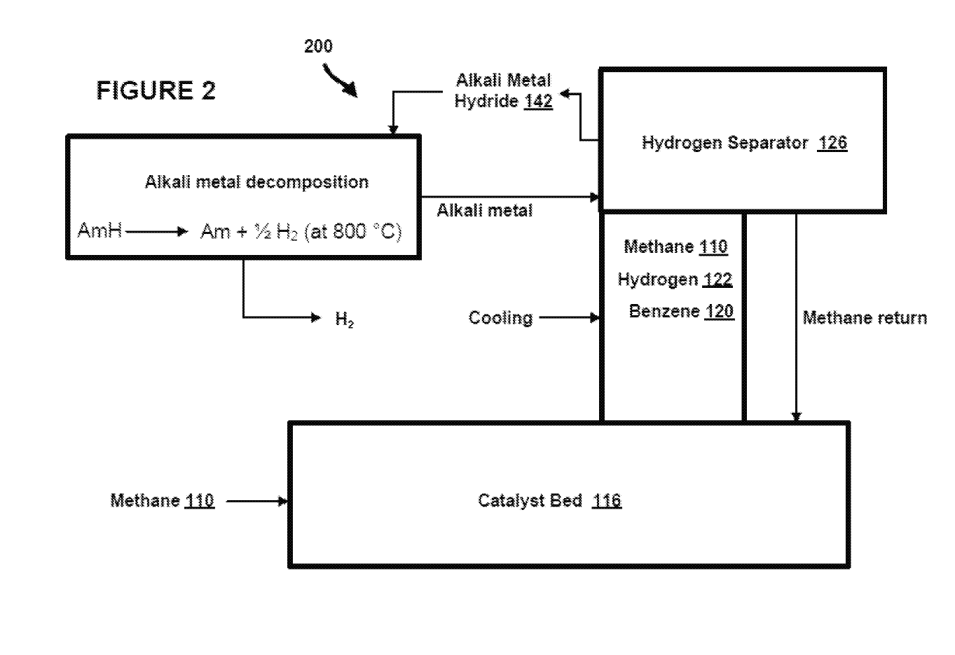

Referring now to FIG. 2, another system 200 for hydrogen separation is shown. The system 200 is similar to that which was disclosed in conjunction with FIG. 1. Accordingly, for purposes of brevity, much of the above-recited description will be omitted.

As shown in FIG. 2, once the products (e.g., the hydrogen 122, benzene 120 and unreacted methane 110 leave the catalyst bed 116, these materials may be subjected to a cooling device. This cooling device operates to cool the benzene 120 such that it returns to its liquefied state, and thus falls back into the catalyst bed. In other words, the benzene 120 that is produced is “refluxing.” At the same time, hydrogen 122 and unreacted methane 110 remain in their gaseous form, and thus enter the hydrogen separator 126. (In some embodiments, the hydrogen and/or methane may have to be heated back up to a higher temperature before entering the separator 126.) In the hydrogen separator 126, the hydrogen 122 is reacted with an alkali metal to produce an alkali metal hydride 142 that is separated out, while the unreacted methane may return to the catalyst bed 116.

As noted above, any technique that is capable of separating out the hydrogen from the other reaction products (so that this hydrogen may be removed to drive the reaction) may be used. For example, embodiments may be constructed in which the hydrogen gas is removed based upon it being a different density and/or having a different diffusion rate (flow rate) than the benzene and/or the methane. If density is used to separate out the gases, the lighter gases will flow to the top of the vessel for collection and separation while the heavier gases will collect at the bottom of the vessel. Those skilled in the art will appreciate how to construct these chambers/vessels as a means of separating out the hydrogen gas from the benzene and/or methane.

FIG. 2 also shows the optional step of alkali metal decomposition. Specifically, the alkali metal hydride (designated “AmH” may be heated to a temperature greater than or equal to 800° C. such that the alkali metal (Am) and hydrogen gas is regenerated. The alkali metal may then be re-added to the separator 126 and the hydrogen gas collected.

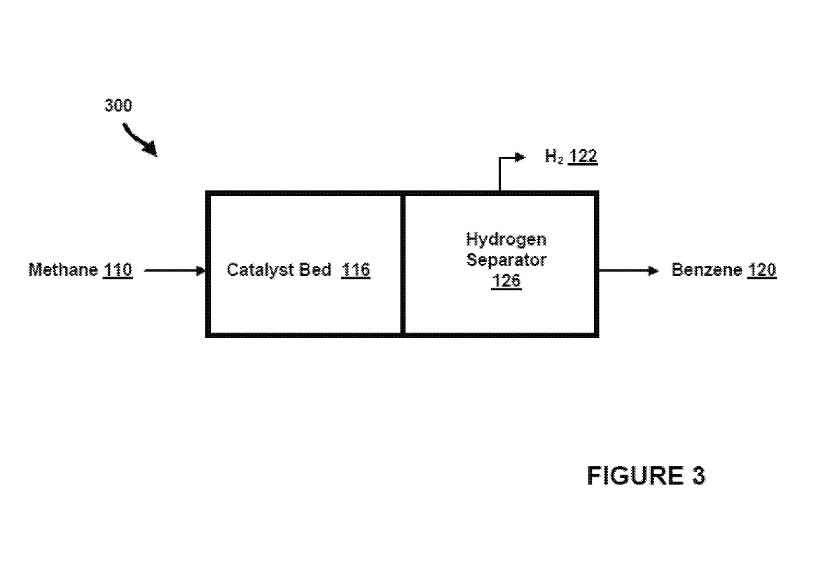

Referring now to FIG. 3, another embodiment of a system 300 for removing hydrogen from a DHA reaction (or other chemical reaction) is illustrated. In the embodiment shown in FIG. 3, the catalyst bed 116 and the hydrogen separator 126 may be in the same vessel. For example, these two features may be in different chambers of the same vessel. In other embodiments, the hydrogen separator 126 may be in the same chamber as the catalyst bed 116. For example, the hydrogen separator 126 may comprise an alkali metal or an alloy of an alkali metal (such as Li—Al) that does not interfere or poison the catalysts in the catalyst bed 116. Thus, once formed from the reaction at the catalyst bed 116, the hydrogen may then immediately react with the alkali metal or alkali metal alloy (in the same chamber) to remove the hydrogen and further drive the production of benzene. The hydrogen 122 is shown being removed from the chamber in FIG. 3.

It is to be understood that the claims are not limited to the precise configuration and components illustrated above. Various modifications, changes and variations may be made in the arrangement, operation and details of the systems, methods, and apparatus described herein without departing from the scope of the claims.

What is claimed is:

1. A method of producing benzene from methane or another organic gas comprising:
conducting a reaction (such as the dehydroaromatization (DHA) reaction) to produce benzene and hydrogen gas from methane; and
removing the produced hydrogen, thereby driving the reaction to produce more benzene.

2. The method of claim 1, wherein the produced hydrogen is removed by reacting the produced hydrogen with an alkali metal or an alloy of an alkali metal, thereby forming an alkali metal hydride.

3. The method of claim 2, wherein the alkali metal hydride is decomposed by heating the material at or above a temperature of 800° C. to generate hydrogen gas and the alkali metal, wherein the alkali metal is re-used to form another batch of the alkali metal hydride.

4. The method of claim 1, wherein the produced hydrogen is removed via a density separation technique or a gas diffusion separation technique.

5. The method of claim 2, wherein the alkali metal is selected from the group consisting of sodium, potassium and lithium.

6. The method of claim 2, wherein the alloy of the alkali metal comprises Li—Al, Li—Si, Na—Al, or Na—Si.
